(19) United States
(12) Patent Application Publication
(10) Pub. No.: US 2016/0331571 A1
(43) Pub. Date: Nov. 17, 2016

(54) LATERAL SUPPORT SYSTEM FOR FEET

(71) Applicant: Donald SPECTOR, New York, NY (US)

(72) Inventor: Donald SPECTOR, New York, NY (US)

(21) Appl. No.: 14/987,173

(22) Filed: Jan. 4, 2016

Related U.S. Application Data
(63) Continuation-in-part of application No. 14/795,151, filed on Jul. 9, 2015.
(60) Provisional application No. 62/160,102, filed on May 12, 2015.
(60) Provisional application No. 62/246,343, filed on Oct. 26, 2015.

(51) Int. Cl. A61F 5/01; A43B 17/00

(52) U.S. Cl. CPC A61F 5/0127; A43B 17/003

(57) ABSTRACT

A stability system is formed from a bottom platform having a top surface configured to receive a sole of a shoe, an interior side and an exterior side. A leg cuff is connected to the exterior side of the bottom platform and comprises a vertical sidewall configured to abut a user's leg when the user's foot is placed on the bottom platform. The leg cuff has an intermediate portion extending around a rear of the stability system and being connected to the bottom platform near an interior side of the bottom platform, to secure the user's heel in place. The stability system is configured such that when a user places a shoe onto the bottom platform, the bottom platform extends beyond a width of the shoe at least on the exterior side and the intermediate portion surrounds the user's ankle. The extra width provides additional stability to a user whose balance is compromised.

### CROSS REFERENCE TO RELATED APPLICATIONS

This is a continuation-in-part of U.S. patent application Ser. No. 14/795,151, filed on Jul. 9, 2015, which claims prioirty under 35 U.S.C. §119(e) of U.S. Provisional Patent Application Ser. No. 62/160,102, filed on May 12, 2015. Applicant also claims priority under 35 U.S.C. §119(e) of U.S. Provisional Patent Application Ser. No. 62/246,343 filed Oct. 26, 2015. The disclosures of both of these applications are hereby incorporated by reference.

### BACKGROUND OF THE INVENTION

1. Field of the Invention

The present invention relates to a lateral support system for attachment to shoes or the legs of a person in conjunction with custom shoe inserts that compensate for medical conditions that reuqire the user to use the shoe stabilizers.

2. The Prior Art

People who have mobility issues often use a cane or a walker to assist in walking from place to place. These objects are bulky, cumbersome and require the use of at least one hand, and are thus inconvenient to use. It would be desirable to provide a stability system that does not require the use of the person's hands and which is not bulky or cumbersome.

### SUMMARY OF THE INVENTION

It is therefore an object of the invention to provide a stability system that allows the user to have both hands free. It is another object of the invention to provide a stability system that is unobtrusive and easy to use. It is another object of the invention to provide a system that uses both custom shoe inserts as well as exterior stabilizers to give the wearer the optimum comfort and stability.

These and other objects are accomplished by a stability system comprising a bottom platform having a top surface configured to receive a sole of a shoe, an interior side and an exterior side. A leg cuff is connected to the exterior side of the bottom platform and comprises a vertical sidewall configured to abut a user's leg when the user's foot is placed on the bottom platform. The leg cuff has an intermediate portion extending around a rear of the stability system and being connected to the bottom platform near an interior side of the bottom platform, to secure the user's heel in place.

The stability system is configured such that when a user places a shoe onto the bottom platform, the bottom platform extends beyond a width of the shoe at least on the exterior side and the intermediate portion surrounds the user's ankle. The extra width provides additional stability to a user whose balance is compromised. The added width is compensated by a thickened region fo the vertical sidewall, so that the bottom surface of the bottom portion is wider, preferably by at least ½ inch, than the top surface that receives the shoe.

The vertical sidewall of the leg cuff is curved laterally to approximate a countour of a user's leg. The bottom platform does nt extend the entire lengt of the user's foot so that the intermediate portion extends beyond the rear of the bottom platform and is not connected to the rear of the bottom platform, so as to create an opening for a heel of the user's shoe. These measures increase the user's comfort while wearing the stability system.

To keep the stability system in place, there is at least one securing strap connected to at least one of the leg cuff and bottom platform for securing the stability system to a user's leg or foot. Any type of strap and attachment mechanism could be used, such as buckles, snaps or a hook-and-loop type closure (VELCRO®)

The leg cuff preferably has an extension that extends along the exterior side of the bottom platform toward a front of the bottom platform so that the bottom platform is securely connected to the cuff and moves with the user. Because many people with balance problems tend to fall forward, a lift mechanism can be disposed on a front of this extension, for lifting the front of the stability system and thus the user's foot slightly off of the ground. Raising the front of the foot moves the user's weight backward slightly, so that forward falls are minimized. The lift mechanism can be formed of a lever having a raised and lowered position, wherein in the lowered position, the lever extends below the bottom surface of the bottom platform. Thus when the user steps down, the lever contacts the ground and keeps the user's toes slightly off of ground level. The lever can be lockable in the lowered position during use, and be moved to a raised position when needed.

To increase the attachment of the leg cuff to the user, the leg cuff can have a top portion that extends around to an interior side of the stability system, so that it wraps around the user's calf.

The bottom is preferably equipped with treads on the bottom surface to prevent the user from slipping.

The stability system also includes a custom fabricated shoe insert that is placed inside the shoe and is used in conjunction with the stability system to allow the user to walk more comfortably.

The shoe insert is created by scanning the user's foot, analyzing the foot anatomy via a software program to determine problem areas, coordinating this analysis with the user's gait in the stability system and creating a custom orthotic via 3D printing to adjust the user's stance and gait. The insert is preferably formed of a compressible material, such as foam or gel, so that the insert can cushion the user's gait. The insert can also be made to include protrusions to take the space of missing toes, thus increasing the stability of the system during use.

The stabilizer and the insert can be created in one step usinga 3D printer, which can use data from scanning the user's anatomy to create a truly custom foot stabilization system that can compensate for a variety of anatomical defects and balance problems.

### BRIEF DESCRIPTION OF THE DRAWINGS

Other objects and features of the present invention will become apparent from the following detailed description considered in connection with the accompanying drawings. It is to be understood, however, that the drawings are designed as an illustration only and not as a definition of the limits of the invention.

In the drawings, wherein similar reference characters denote similar elements throughout the several views:

FIG. 1 shows a side view of the stability system according to the invention on a user's foot;

FIG. 2 shows a rear view of the stability system;

FIG. 3 shows another side view of the stability system;

FIG. 4 is a front view of the stability system;

FIG. 5 is another rear view of the stability system;

FIG. 6 is a bottom view of the stability system;

FIGS. 7a and 7b show of another embodiment of the stability system;

FIG. 8 is a top view of the shoe insert for use in the stability system;

FIG. 9 is a side view of the shoe insert; and

FIG. 10 shows the stability system in use.

### DETAILED DESCRIPTION OF THE PREFERRED EMBODIMENT

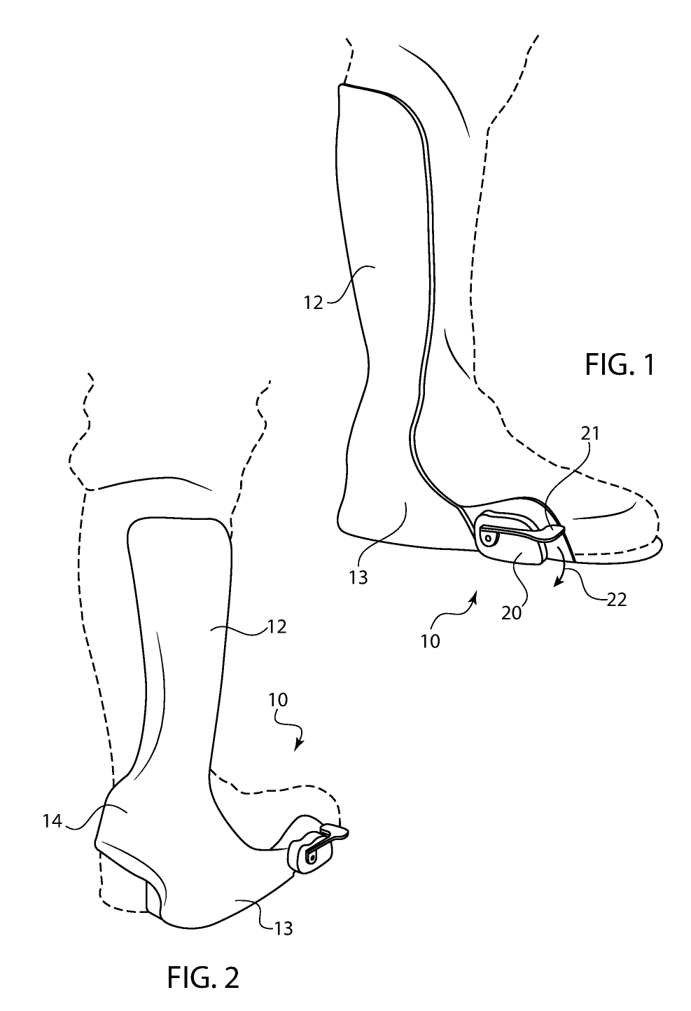

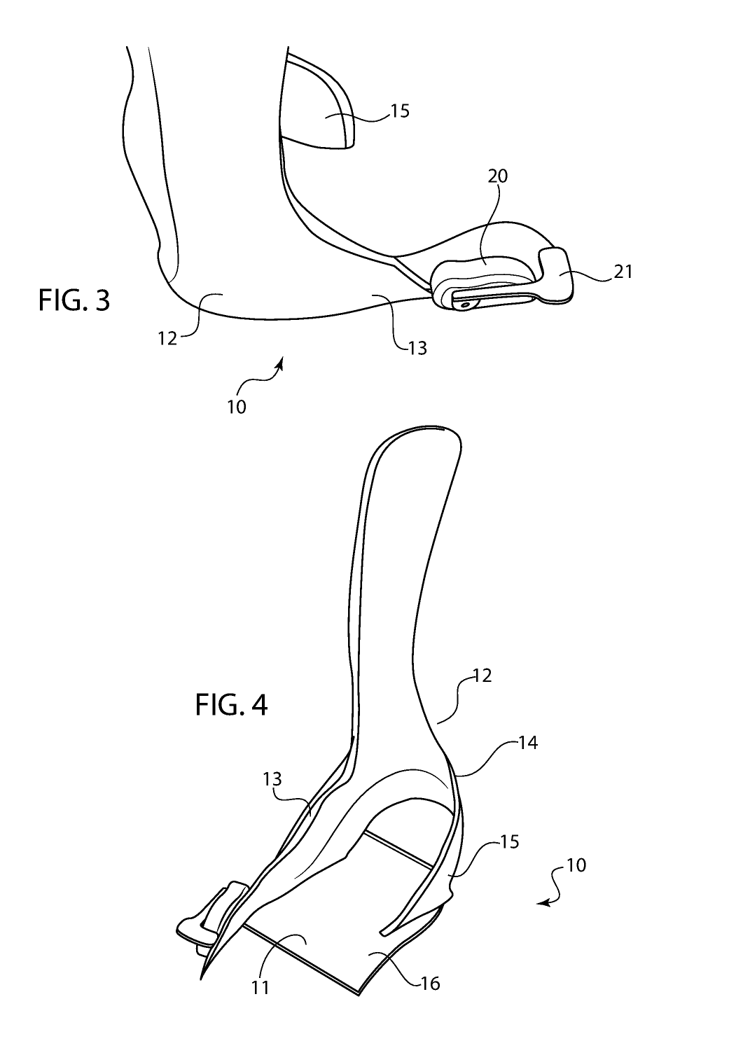

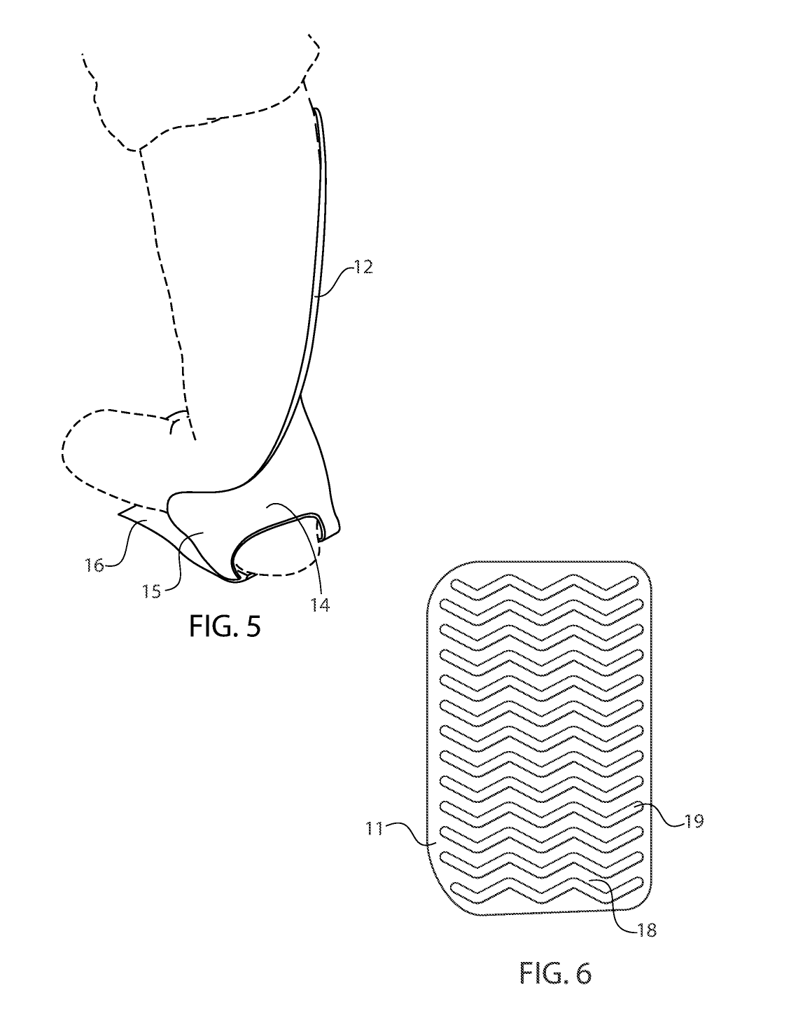

Referring now in detail to the drawings and, in particular, FIGS. 1-5 show one embodiment of the shoe stabilizer 10 according to the invention. Stabilizer 10 is formed by a bottom platform 11 having a top surface 16 connected to a leg cuff 12. Leg cuff 12 has a lateral extension 13 connected to the exterior side of the bottom portion, an intermediate heel portion 14 extending around the rear of the shoe stabilizer, and an interior portion 15 connected to the interior side of the bottom platform 11. The lateral extension 13 is thickened especially in the area connected to bottom platform 11, so that bottom platform 11 is wider than the exposed top surface 16 that can accommodate a shoe. This extra width adds stability to a user when walking, as a greater surface area is contacting the ground.

In addition, interior portion 15 can be connected to a portion of the top surface 16 disposed inwardly of the inside edge, creating another section of the bottom platform that extends beyond the width of the user's shoe.

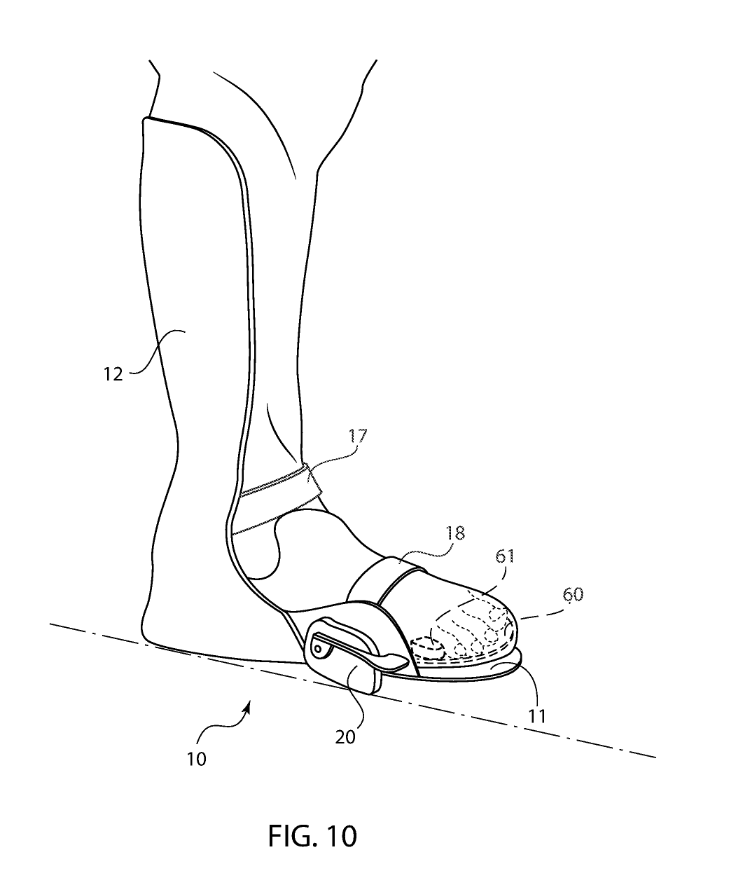

As shown in FIG. 1, lateral extension 13 can be equipped with a lift 20 that can be rotated downward in the direction of arrow 22 by pressing on lever 21. This causes the lift 20 to be positioned below the level of the bottom platform, so that when the user steps down, the front section of the bottom platform is lifted off of the ground, such as shown in FIG. 10. This tilt causes the user's weight to be moved rearward and works to prevent forward falls.

As shown in FIG. 6, the bottom surface 18 of bottom platform 11 can be equipped with treads to prevent the user from slipping on various surfaces.

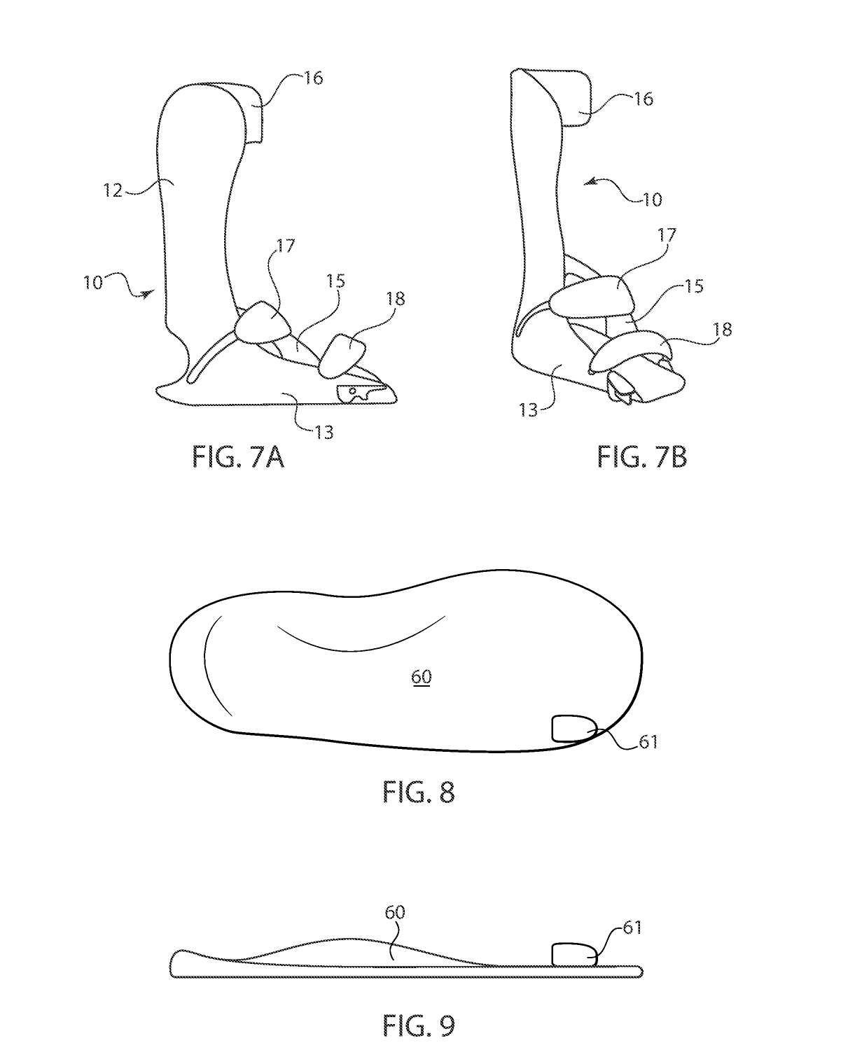

An alternative embodiment is shown in FIGS. 7a and 7b. In FIGS. 7a and 7b, leg cuff 12 is equipped with an upper extension 16 that curves around the rear of the stabilizer to the interior side. Extension 16 is configured to wrap around the calf of the user, to keep stabilizer 10 in place. Straps 17, 18 can be attached to the stabilizer 10 at various locations, such as across the front or ankle area to secure stabilizer 10 to the user.

FIGS. 8 and 9 show the orthopedic insert 60 according to the invention. Insert 60 is in the form of a footbed that is placed in the bottom of the user's shoe while the user is using stabilizer 10. Insert 60 is made of a compressible material such as foam or gel, that adds extra cushioning to the foot while walking with stabilizer 10. For people who have lost a toe, such as from diabetes, insert 60 can be manufactured with a protrusion 61 to take the space of the missing toe in the shoe. This adds extra stability to the shoe and works in conjuction with stabilizer 10 as shown in FIG. 10 to prevent the user from falling.

The shoe insert is created by scanning the user's foot, analyzing the foot anatomy via a software program to determine problem areas, coordinating this analysis with the user's gait in the stability system and creating a custom orthotic via 3D printing to adjust the user's stance and gait.

Accordingly, while only a few embodiments of the present invention have been shown and described, it is obvious that many changes and modifications may be made thereunto without departing from the spirit and scope of the invention.

What is claimed is:

1. A stability system comprising:
a bottom platform configured to receive a sole of a shoe, the bottom platform having a top surface, a bottom surface, a front, a rear, an exterior side and an interior side;
a leg cuff connected to the exterior side of the bottom platform and extending up from the top surface, the leg cuff comprising a vertical sidewall configured to abut a user's leg when the user's foot is placed on the bottom platform, the leg cuff having an intermediate portion extending around a rear of the stability system and being connected to the bottom platform near an interior side, wherein the leg cuff has a thickened region along the exterior side such that the bottom surface of the bottom platform has a width that is greater than the top surface of the bottom platform.

2. The stability system according to claim 1, wherein the vertical sidewall is curved laterally to approximate a countour of a user's leg.

3. The stability system according to claim 1, wherein the intermediate portion extends beyond the rear of the bottom platform and is not connected to the rear of the bottom platform, so as to create an opening for a heel of the user's shoe.

4. The stability system according to claim 1, further comprising at least one securing strap connected to at least one of the leg cuff and bottom platform for securing the stability system to a user's leg or foot.

5. The stability system according to claim 1, wherein the leg cuff has an extension that extends along the exterior side of the bottom platform toward a front of the bottom platform, and further comprising a lift mechanism disposed on a front of the extension, the lift mechanism comprising a lever having a raised and lowered position, wherein in the lowered position, the lever extends below the bottom surface of the bottom platform.

6. The stability system according to claim 5, wherein the lever is lockable in the lowered position.

7. The stability system according to claim 1, wherein the leg cuff has a top portion that extends around to an interior side of the stability system.

8. The stability system according to claim 1, wherein the bottom platform is equipped with treads on the bottom surface.

9. The stability system according to claim 1, further comprising an insert in the form of a footbed configured to be placed inside a shoe of the user.

10. The stability system according to claim 9, wherein the insert is made of foam or gel.

11. The stability system according to claim 9, wherein the insert contains a protrusion in a front area, said protrusion filling space within the shoe to compensate for a missing toe.

12. The stability system according to claim 1, wherein the width of the bottom surface of the bottom platform is at least ½ inch greater than the width of the top surface of the bottom platform.
